(19) United States
(12) Patent Application Publication
(10) Pub. No.: US 2017/0316176 A1
(43) Pub. Date: Nov. 2, 2017

(54) DEVICE FOR ANALYZING INSULIN SECRETION ABILITY, SYSTEM FOR ANALYZING INSULIN SECRETION ABILITY PROVIDED WITH SAME, AND METHOD FOR ANALYZING INSULIN SECRETION ABILITY

(71) Applicant: HITACHI, LTD., Tokyo (JP)

(72) Inventors: Yasutaka HASEGAWA, Tokyo (JP); Hideyuki BAN, Tokyo (JP); Takashi WADA, Tokyo (JP)

(21) Appl. No.: 15/517,642

(22) PCT Filed: Dec. 25, 2014
(86) PCT No.: PCT/JP2014/084285

§ 371 Date: Apr. 7, 2017

(51) Int. Cl. G06F 19/00; A61B 5/145

(52) U.S. Cl. CPC G06F 19/3443; G06F 19/3437; A61B 5/14532; G06F 19/3481

(57) ABSTRACT

The present invention enables evaluation of insulin secretion ability by a simpler method. A device for analyzing insulin secretion ability that comprises: an input unit inputting at least a fasting blood sugar level and an HbA1c level; an estimated HbA1c calculation unit calculating an estimated HbA1c level from the inputted fasting blood sugar level and HbA1c level; and an evaluated insulin secretion ability calculation unit calculating an evaluated insulin secretion ability on the basis of the HbA1c level inputted by the input unit and the estimated HbA1c level calculated by the estimated HbA1c calculation unit.

### TECHNICAL FIELD

The present invention relates to an analysis device for analyzing an insulin secretory capacity of a subject to be analyzed, an analysis system including the analysis device, and an analysis method.

### BACKGROUND ART

Insulin is secreted from the pancreas and works to adjust a blood glucose level. Diabetes is diagnosed by a diabetes type based on any blood glucose level among fasting blood glucose level, casual blood glucose level, and blood glucose level two hours after 75-g oral glucose tolerance test (OGTT), or a hemoglobin A1c (HbA1c) value. That is, when a diabetes type is confirmed twice or more in the medical tests which are performed on the examinee on different days, the examinee is diagnosed as diabetes. Diabetes progresses with less subjective symptom and leads to a serious complication such as nephropathy, and thus a treatment therefor is important.

It is known that an insulin secretory capacity has a great effect on pathogeny and progress of diabetes, and it is necessary to evaluate the insulin secretory capacity of an examinee for the diabetes treatment. Conventionally, there has been known an insulinogenic index calculated from an insulin concentration in the blood and a value of the glucose tolerance test as an insulin secretory capacity evaluation index. The insulinogenic index is calculated in the following equation. Insulinogenic index=(insulin concentration 30 minutes after glucose tolerance test−fasting insulin concentration)/(blood glucose level 30 minutes after glucose tolerance test−fasting blood glucose level). As the value is lower, the insulin secretory capacity is lower, and when the value is 0.4 or less, the insulin secretory capacity is determined as incomplete (low).

PTL 1 discloses a diabetes diagnosis support system for analyzing a clinical condition of diabetes of a patient and outputting diagnosis support information such as exercise therapy and diet on the basis of the laboratory data and clinical presentation of the patient. The system disclosed in PTL 1 is directed for outputting diagnosis support information on diabetes by use of a reduction in insulin secretory capacity as one index on the basis of the input values of fasting insulin value, blood glucose level, insulin value after glucose tolerance test, and the like. The system disclosed in PTL 1 determines an insulin secretory capacity on the basis of the input fasting insulin value and insulin value after glucose tolerance test.

### CITATION LIST

### Patent Literature

PTL 1: Publication of Patent US2004/0091424

### SUMMARY OF INVENTION

### Technical Problem

The insulin concentration for evaluating an insulin secretory capacity is generally measured by use of insulin antibody in the chemiluminescence immunoassay method (CLIA method). The insulin concentration measurement is not an item to be examined for general medical checkup or complete medical checkup.

That is, the insulin concentration, which is not measured in general medical checkup or complete medical checkup, generally needs to be measured while the subject is hungry and after the glucose tolerance test in order to evaluate the insulin secretory capacity, not only in the diabetes diagnosis support system. Thus, there is conventionally a problem that it takes much time, cost and steps for evaluating the insulin secretory capacity.

It is therefore an object of the present invention to provide an insulin secretory capacity analysis device capable of evaluating an insulin secretory capacity in a simpler method than before, an insulin secretory capacity analysis system including the device, and an insulin secretory capacity analysis method.

### Solution to Problem

In order to achieve the object, the present inventors have progressively studied, and have found that an insulin secretory capacity can be calculated by use of a fasting blood glucose level and a hemoglobin A1c value (HbA1c value), and have completed the present invention. The present invention encompasses the followings.

(1) An insulin secretory capacity analysis device including: an input unit that inputs at least a fasting blood glucose level and an HbA1c value; an estimated HbA1c calculation unit that calculates an estimated HbA1c value on the basis of the input fasting blood glucose level and HbA1c value; and an insulin secretory capacity evaluation value calculation unit that calculates an insulin secretory capacity evaluation value on the basis of the HbA1c value input by the input unit and the estimated HbA1c value calculated by the estimated HbA1c calculation unit.

(2) The insulin secretory capacity analysis device according to (1), wherein the estimated HbA1c calculation unit calculates an estimated HbA1c value on the basis of the input fasting blood glucose level and HbA1c value by use of a relational equation of the fasting blood glucose level and the HbA1c value created on the basis of a dataset including the fasting blood glucose level and the HbA1c value of a plurality of examinees.

(3) The insulin secretory capacity analysis device according to (2), wherein the regression analysis is made assuming the HbA1c value as objective variable and the fasting blood glucose level as explanatory variable thereby to create the relational equation.

(4) The insulin secretory capacity analysis device according to (1), wherein the insulin secretory capacity evaluation value calculation unit calculates the insulin secretory capacity evaluation value on the basis of a difference between the HbA1c value input by the input unit and the estimated HbA1c value calculated by the estimated HbA1c calculation unit.

(5) The insulin secretory capacity analysis device according to (1), further including: an output unit that outputs information on an insulin secretory capacity by comparing the insulin secretory capacity evaluation value calculated by the insulin secretory capacity evaluation value calculation unit with a reference value.

(6) The insulin secretory capacity analysis device according (1), further including: a management necessity determination unit that determines the necessity of management of diabetes on the basis of weight information input by the input unit and the insulin secretory capacity evaluation value calculated by the insulin secretory capacity evaluation value calculation unit.

(7) The insulin secretory capacity analysis device according to (1), further including: a medical checkup data storage unit that stores therein a dataset including a fasting blood glucose level and an HbA1c value of a plurality of examinees; and a subject-of-management selection unit that selects a subject of management of diabetes from among the dataset stored in the medical checkup data storage unit on the basis of the weight information input by the input unit and the insulin secretory capacity evaluation value calculated by the insulin secretory capacity evaluation value calculation unit.

(8) An insulin secretory capacity analysis system including: the insulin secretory capacity analysis device according to any one of the above described (1) to (7); and a terminal having a dataset including at least a fasting blood glucose level and an HbA1c value of a subject to be analyzed, wherein the dataset of a subject to be analyzed is input from the terminal into the insulin secretory capacity analysis device, and an insulin secretory capacity of the subject to be analyzed is analyzed by the insulin secretory capacity analysis device.

(9) The insulin secretory capacity analysis system according to (8), wherein the terminal is a measurement instrument for measuring a fasting blood glucose level of a subject to be analyzed and/or for measuring an HbA1c value.

(10) An insulin secretory capacity analysis method including the steps of: inputting a fasting blood glucose level and an HbA1c value; calculating an estimated HbA1c value on the basis of the input fasting blood glucose level and HbA1c value; and calculating an insulin secretory capacity evaluation value on the basis of the input HbA1c value and the calculated estimated HbA1c value.

### Advantageous Effects of Invention

With the insulin secretory capacity analysis device and the insulin secretory capacity analysis method according to the present invention, the insulin secretory capacity of a subject to be analyzed is analyzed on the basis of the fasting blood glucose level and the HbA1c value of the subject to be analyzed. Thus, the insulin secretory capacity analysis device according to the present invention can acquire information on the insulin secretory capacity much more simply and at lower cost than ever.

Further, with the insulin secretory capacity analysis system according to the present invention, the insulin secretory capacity of a subject to be analyzed is analyzed by the insulin secretory capacity analysis apparats on the basis of the fasting blood glucose level and the HbA1c value of the subject to be analyzed input from a terminal. Thus, the insulin secretory capacity analysis system according to the present invention can acquire information on the insulin secretory capacity much more simply and at lower cost than ever.

### BRIEF DESCRIPTION OF DRAWINGS

FIG. 1 is a block diagram illustrating an exemplary configuration of an insulin secretory capacity analysis device to which the present invention is applied.

FIG. 2 is a block diagram illustrating other exemplary configuration of the insulin secretory capacity analysis device to which the present invention is applied.

FIG. 3 is a diagram illustrating exemplary medical checkup data.

FIG. 4 is a flowchart illustrating an exemplary relational equation creation processing.

FIG. 5 is a diagram illustrating exemplary relational equation data.

FIG. 6 is a diagram illustrating an exemplary screen for confirming relational equations.

FIG. 7 is a flowchart illustrating an exemplary insulin secretory capacity evaluation equation creation processing.

FIG. 8 is a diagram illustrating exemplary insulin secretory capacity evaluation equation data.

FIG. 9 is a flowchart illustrating an exemplary insulin secretory capacity evaluation processing.

FIG. 10 is a diagram illustrating exemplary results of an insulin secretory capacity evaluation value evaluated by an insulinogenic index.

FIG. 11 is a diagram illustrating exemplary results of an insulin secretory capacity evaluation value evaluated by a risk of diabetes.

FIG. 12 is a flowchart illustrating an exemplary subject-of-management selection processing.

FIG. 13 is a diagram illustrating an exemplary subject-of-management selection screen.

FIG. 14 is a diagram illustrating exemplary results of a combination of insulin secretory capacity evaluation value and weight change evaluated by a risk of diabetes.

FIG. 15 is a diagram illustrating other exemplary insulin secretory capacity evaluation equation data.

### DESCRIPTION OF EMBODIMENTS

An embodiment of the present invention will be described below in detail with reference to the drawings.

An insulin secretory capacity analysis device according to the present invention is directed for previously measuring a fasting blood glucose level and an HbA1c value of a blood specimen taken from a subject to be analyzed, and analyzing an insulin secretory capacity of the subject to be analyzed by use of the fasting blood glucose level and the HbA1c value. Herein, a subject to be analyzed is not particularly limited, and means a human being. The subject to be analyzed may be an examinee of medical checkup, a patient of diabetes (including type I and type II), and a person with suspected diabetes. The insulin secretory capacity of the subject to be analyzed is analyzed thereby to provide information on a change (particularly reduction) in insulin secretory capacity, such as to know the probability of diabetes of the subject to be analyzed, to support the diagnosis of diabetes of the subject to be analyzed, to provide information for selecting a diabetes treatment of the subject to be analyzed, and to support an improvement of living for preventing diabetes of the subject to be analyzed.

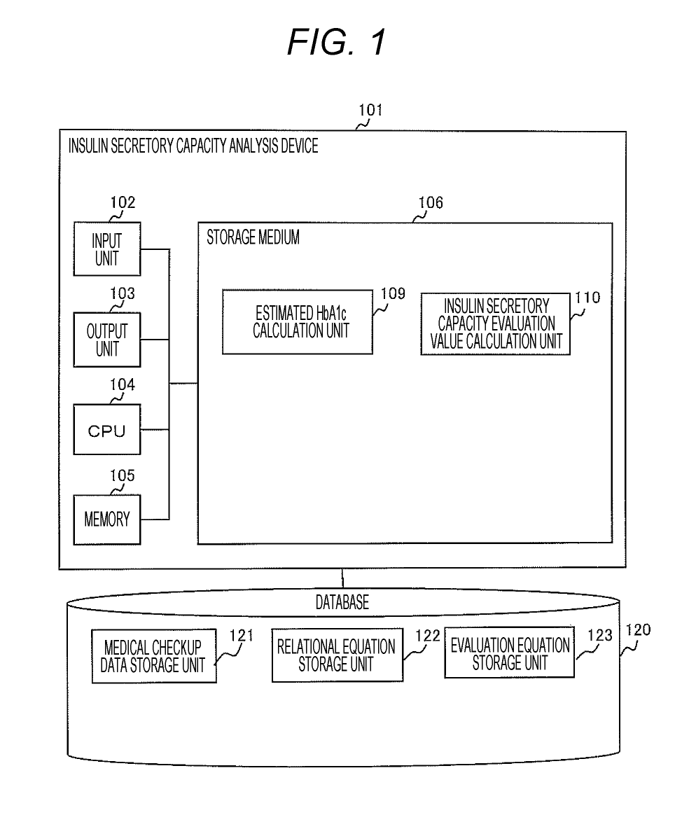

More specifically, as illustrated in FIG. 1, an insulin secretory capacity analysis device 101 to which the present invention is applied includes an input unit 102 for inputting at least a fasting blood glucose level and an HbA1c value, an estimated HbA1c calculation unit 109 for calculating an estimated HbA1c value on the basis of the fasting blood glucose level and the HbA1c value input in the input unit 102, and an insulin secretory capacity evaluation value calculation unit 110 for calculating an insulin secretory capacity evaluation value on the basis of the HbA1c value input in the input unit 102 and the estimated HbA1c value calculated in the estimated HbA1c calculation unit 109. The insulin secretory capacity analysis device 101 includes an output unit 103 for outputting a result or the like of the analyzed insulin secretory capacity, a CPU 104 for executing various information processing programs, a memory 105 for developing an information processing program to be executed or data used by an information processing program, and a storage medium 106 for storing therein information processing programs such as the estimated HbA1c calculation unit 109 and the insulin secretory capacity evaluation value calculation unit 110.

The insulin secretory capacity analysis device 101 illustrated in FIG. 1 may be configured as an insulin secretory capacity analysis system which is connected to an external database 120 thereby to acquire the relational equations used by the estimated HbA1c calculation unit 109 and the evaluation equations used by the insulin secretory capacity evaluation value calculation unit 110 from the database 120. The relational equations used by the estimated HbA1c calculation unit 109 and/or the evaluation equations used by the insulin secretory capacity evaluation value calculation unit 110 may be stored in the storage medium 106 and read from the storage medium 106 for use, not limited to being in the external database 120.

In the insulin secretory capacity analysis device 101, the input unit 102 may be a human interface such as mouse or keyboard, and is directed for receiving the entry into the insulin secretory capacity analysis device 101. Further, the input unit 102 may employ an input device capable of inputting the blood analysis results of a subject to be analyzed, such as fasting blood glucose level and HbA1c value. The input unit 102 may be a network interface capable of inputting information into a terminal storing the blood analysis results of the subject to be analyzed via a network, or may be an interface such as USB which is mounted with a measurement instrument for analyzing the blood of a subject to be analyzed and inputting information from the measurement instrument.

The output unit 103 may be a display or printer for outputting a calculation result by the insulin secretory capacity analysis device 101. Further, the output unit 103 may be an interface for outputting an insulin secretory capacity evaluation value calculated by the insulin secretory capacity evaluation value calculation unit 110 to an external terminal.

The storage medium 106 is a storage device, such as nonvolatile storage medium (including magnetic disc drive or nonvolatile memory), for storing therein various programs for realizing an insulin secretory capacity analysis processing by the insulin secretory capacity analysis terminal 101 and the results of the insulin secretory capacity analysis processing.

The CPU 104 is a computing device, such as CPU or GPU, for executing the programs loaded into the memory 105. The processing and calculations described below are performed by the CPU 104.

The insulin secretory capacity analysis device 101 is a computer system configured on one computer or a plurality of logically or physically configured computers, and may operate in different threads on the same computer or may operate on a virtual computer constructed on a plurality of physical computer resources.

The programs executed by the CPU 104 may be provided to each server via a removable medium (such as CD-ROM or flash memory) or a network to be stored in a nonvolatile storage device as non-transitory storage medium. In this case, the insulin secretory capacity analysis device 101 may include an interface for reading a removable medium.

In the thus-configured insulin secretory capacity analysis device 101, a relational equation used by the estimated HbA1c calculation unit 109 means a relational equation for statistically processing a relationship between the fasting blood glucose level and the HbA1c value included in the medical checkup data of a plurality of examinees and calculating an estimated HbA1c value on the basis of the fasting blood glucose level as described below in detail. An evaluation equation for calculating an insulin secretory capacity evaluation value means an equation for calculating an evaluation value for evaluating an insulin secretory capacity on the basis of an actual HbA1c value and an estimated HbA1c value of a subject to be analyzed. The medical checkup data of the examinees, which is used for creating a relational equation used by the estimated HbA1c calculation unit 109, may include the medical checkup data of the subject to be analyzed.

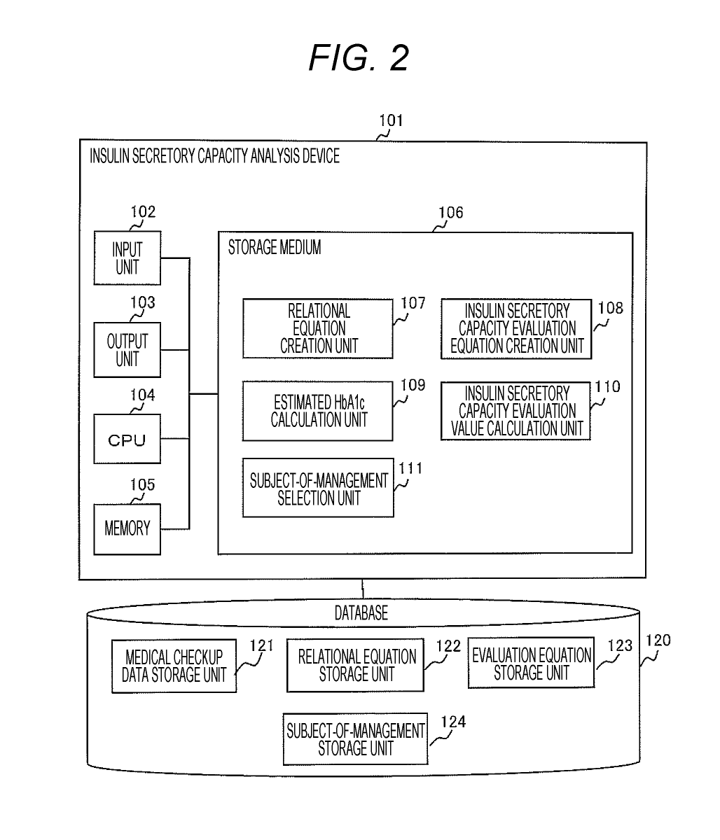

The insulin secretory capacity analysis device 101 illustrated in FIG. 1 is configured to acquire the relational equations and/or the evaluation equations from the external database 120. However, the insulin secretory capacity analysis device according to the present invention is not limited to the configuration, and may be configured to create a relational equation for calculating an estimated HbA1c value and creating an evaluation equation for evaluating an insulin secretory capacity. The insulin secretory capacity analysis device for creating the relational equations and evaluation equations stores a relational equation creation unit 107 and an insulin secretory capacity evaluation equation creation unit 108 in the storage medium 106 in addition to the components illustrated in FIG. 1, for example, as illustrated in FIG. 2. The relational equation creation unit 107 acquires the fasting blood glucose level and the HbA1c value included in the medical checkup data of a plurality of examinees input in the input unit 102, statistically processes a relationship between the HbA1c value and the fasting blood glucose level, and creates a relational equation for calculating an estimated HbA1c value based on the fasting blood glucose level. The insulin secretory capacity evaluation equation creation unit 108 creates an evaluation equation for evaluating an insulin secretory capacity on the basis of the estimated HbA1c value calculated by the relational equation creation unit 107 and the HbA1c value input in the input unit 102.

In the insulin secretory capacity analysis device 101 illustrated in FIG. 2, the estimated HbA1c calculation unit 109 acquires the fasting blood glucose level of the subject to be analyzed input in the input unit 102, and substitutes it into the relational equation created by the relational equation creation unit 107 thereby to calculate an estimated HbA1c value. In the insulin secretory capacity analysis device 101 illustrated in FIG. 2, the insulin secretory capacity evaluation value calculation unit 110 substitutes the HbA1c value of the subject to be analyzed input in the input unit 102 and the estimated HbA1c value calculated by the estimated HbA1c calculation unit 109 into the evaluation equation created by the insulin secretory capacity evaluation equation creation unit 108 thereby to calculate an insulin secretory capacity evaluation value.

The insulin secretory capacity analysis device 101 illustrated in FIG. 2 stores a subject-of-management selection unit 111 in the storage medium 106, and can determine that the subject to be analyzed is in the preliminary step of diabetes, for example, on the basis of the information on the insulin secretory capacity evaluation value calculated by the insulin secretory capacity evaluation value calculation unit 110 and the weight change and can select the subject to be analyzed as a subject of management for prevention of diabetes when the information on the weight change of the subject to be analyzed is input in the input unit 102.

The relational equation creation unit 107 and the insulin secretory capacity evaluation equation creation unit 108 create a relational equation and an evaluation equation, respectively, on the basis of the medical checkup data of a plurality of examinees stored in the database 120. That is, the database 120 includes a medical checkup data storage unit 121 for storing the medical checkup data of a plurality of examinees therein. The database 120 may include a relational equation storage unit 122 for storing the relational equations created by the relational equation creation unit 170 therein, an evaluation equation storage unit 123 for storing the evaluation equations created by the insulin secretory capacity evaluation equation creation unit 108 therein, and a subject-of-management storage unit 124 for storing information on a subject-of-management selected by the subject-of-management selection unit 111 therein.

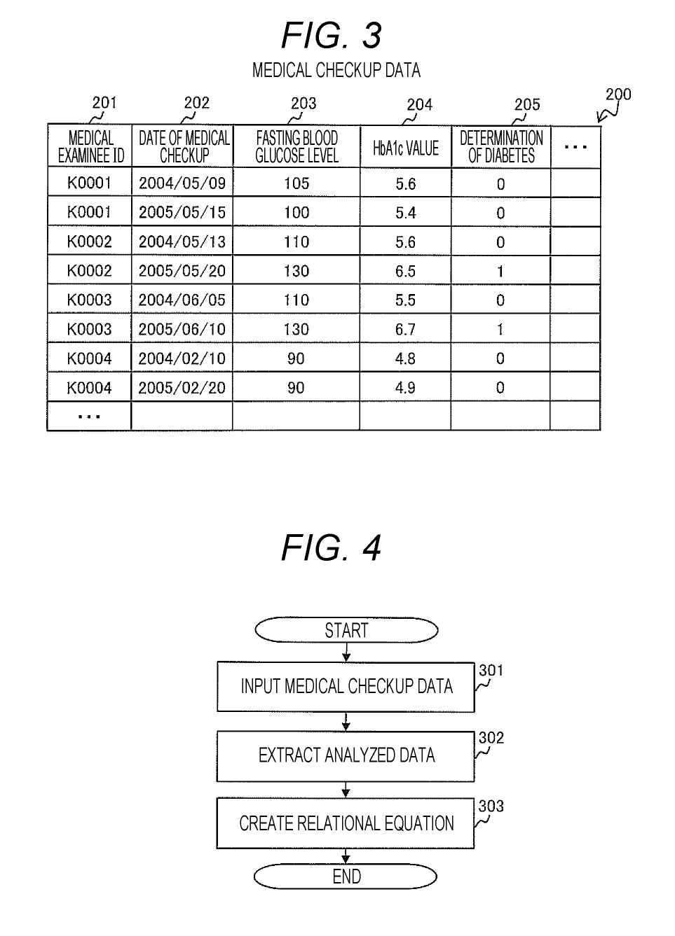

FIG. 3 illustrates a configuration of the medical checkup data stored in the medical checkup data storage unit 121. Medical checkup data 200 includes the medical checkup data of several years of a plurality of examinees. The medical checkup data 200 includes examinee ID 201 assigned to each individual who takes a medical checkup, date of medical checkup 202, fasting blood glucose level 203, HbA1c value 204, determination of diabetes 205, and the like. Examinee ID 201 registers therein the identifier of an examinee who had a medical checkup or complete medical checkup. Date of medical checkup 602 registers therein information on year/month/date when an examinee had a medical checkup or complete medical checkup. Fasting blood glucose level 203 and HbA1c value 204 indicate a fasting blood glucose level and an HbA1c value of an examinee specified by examinee ID 201 which are checked in a medical checkup or complete medical checkup. Fasting blood glucose level 203 indicates a blood glucose level when an examinee is hungry, and is a numerical value which is measured by the defined method and indicated in mg/dl or mol/l. HbA1c value 204 is a value indicating an average blood glucose level of two to three months and is a numerical value in % (JDS value), % (NGSP value) or mmol/mol. Determination of diabetes 205 is a value indicating whether an examinee is under treatment of diabetes, where “1” indicates that an examinee is under treatment and “0” indicates that an examinee is not under treatment. The medical checkup data may include determination of other disease, family history, past medical history, weight, and the like.

A relational equation creation processing by the relational equation creation unit 107 will be described below in the flowchart illustrated in FIG. 4. FIG. 4 is a flowchart in which the relational equation creation unit 107 creates a relational equation of fasting blood glucose level and HbA1c on the basis of the medical checkup data of FIG. 3 by way of example. When the processing in FIG. 4 is started, a medical checkup data input step 301 is first performed.

In the medical checkup data input step 301, the relational equation creation unit 107 acquires the medical checkup data 200 stored in the medical checkup data storage unit 121.

Then in an analysis data extraction step 302, the relational equation creation unit 107 extracts relational equation creation data from the medical checkup data 200 acquired in the medical checkup data input step 301. Specifically, the relational equation creation unit 107 extracts medical checkup data to be analyzed with “0” indicating that the examinee is not under treatment of diabetes in determination of diabetes 205. The fasting blood glucose and HbA1c are influenced by a drug, and thus the medical checkup data to be analyzed, from which the influence is removed, can be extracted.

Then in a relational equation creation step 303, the relational equation creation unit 107 creates a relational equation by use of the medical checkup data to be analyzed extracted in the analysis data extraction step 302. Specifically, the relational equation creation unit 107 statistically processes a relationship between fasting blood glucose level 203 and HbA1c value 204, which is included in the medical checkup data to be analyzed extracted in the analysis data extraction step 302, thereby to create a relational equation for calculating an estimated HbA1c value based on the fasting blood glucose level. Specifically, the regression analysis is made assuming HbA1c value 204 as objective variable and fasting blood glucose level 203 as explanatory variable thereby to create a relational equation. The created relational equation can be stored in the relational equation storage unit 122.

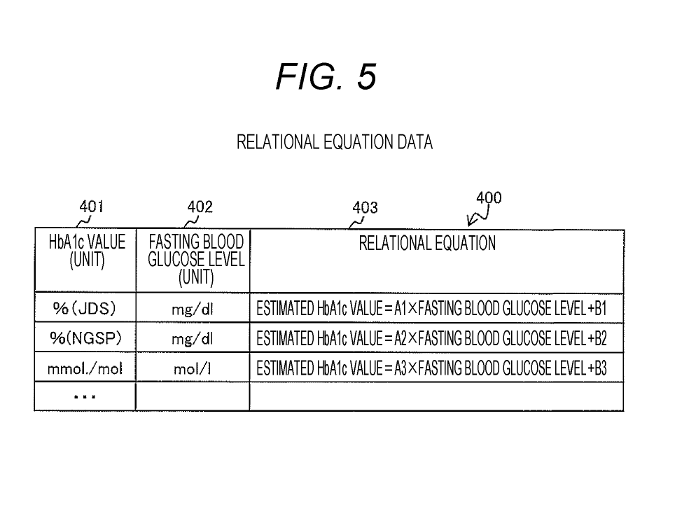

FIG. 5 illustrates exemplary relational equations created by the relational equation creation unit 107. The relational equation data illustrated in FIG. 5 enables an estimated HbA1c value to be calculated on the basis of the fasting blood glucose level of a subject to be analyzed. In relational equation data 400 illustrated in FIG. 5, relational equation 403 is stored per set of the units of HbA1c value 401 and fasting blood glucose level 402. Relational equation 403 indicates a relational equation created by the relational equation creation unit 107, which is described in the form of [estimated HbA1c value]=A1 to A3×fasting blood glucose level +B1 to B3 by way of example. In a relational equation, A1 to A3 and B1 to B3 are a coefficient calculated by the regression analysis per set of the units of HbA1c value 401 and fasting blood glucose level 402.

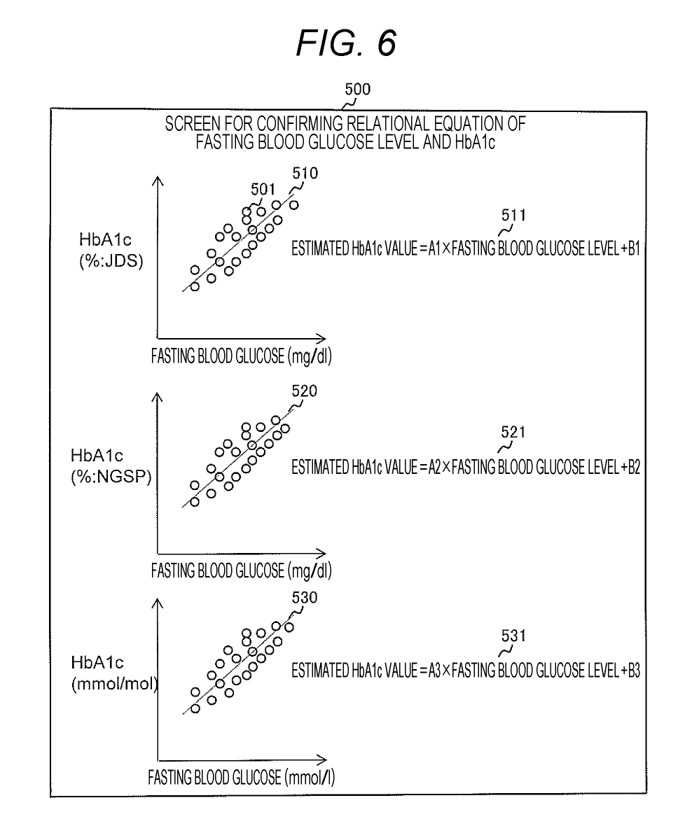

Further, the insulin secretory capacity analysis device 101 can output the relational equations created by the relational equation creation unit 107 as a screen 500 by the output unit 103 as illustrated in FIG. 6. As illustrated in FIG. 6, the output unit 103 can display the relational equations 511, 521, 531 created per combination of the units of HbA1c value 401 and fasting blood glucose level 402 as well as analysis data 501 used for creating the relational equations, and the lines 510, 520, 530 of the relational equations.

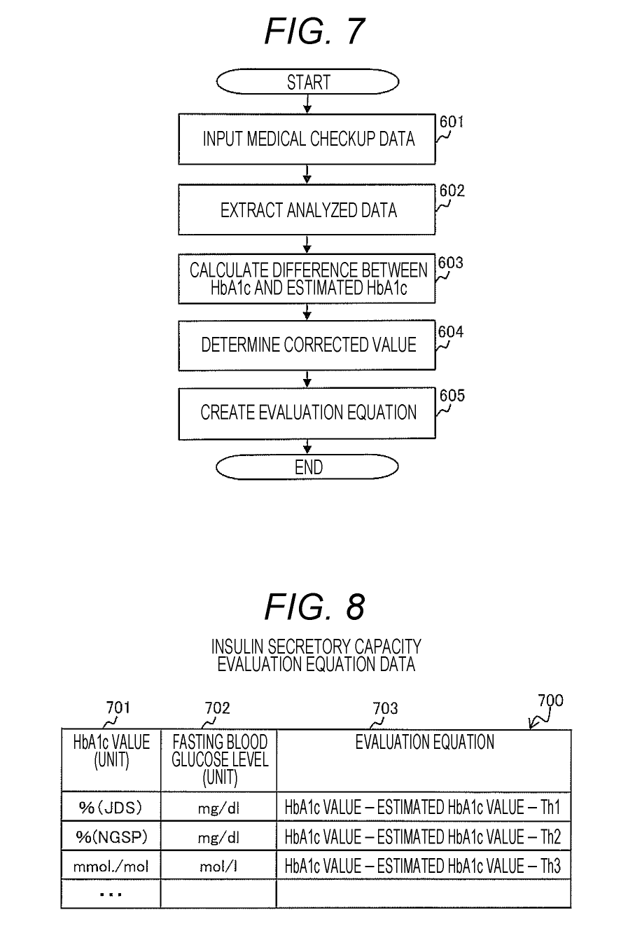

An evaluation equation creation processing by the insulin secretory capacity evaluation equation creation unit 108 will be described below in the flowchart illustrated in FIG. 7. FIG. 7 is an exemplary flowchart in which the insulin secretory capacity evaluation equation creation unit 108 creates an evaluation equation for evaluating an insulin secretory capacity of a subject to be analyzed on the basis of the medical checkup data including the fasting blood glucose level and the HbA1c value illustrated in FIG. 3 and the estimated HbA1c value found based on the relational equation created by the relational equation creation unit 107. When the processing in FIG. 7 is started, a medical checkup data input step 601 is first performed.

In the medical checkup data input step 601, the insulin secretory capacity evaluation equation creation unit 108 acquires the medical checkup data 200 stored in the medical checkup data storage unit 121.

Then in an analysis data extraction step 602, the insulin secretory capacity evaluation equation creation unit 108 extracts evaluation equation creation data from the medical checkup data 200 acquired in the medical checkup data input step 601. Specifically, the insulin secretory capacity evaluation equation creation unit 108 extracts the medical checkup data of two different fiscal years with reference to date of medical checkup 202 per examinee ID 201. For example, the insulin secretory capacity evaluation equation creation unit 108 extracts the medical checkup data with different fiscal years of 2004 and 2009 per examinee ID 201. The insulin secretory capacity evaluation equation creation unit 108 then excludes the medical checkup data with examinee ID 201 of “1” indicating that the examinee is under treatment of diabetes, and extracts evaluation equation creation data with reference to determination of diabetes 205 in the medical checkup data of the older fiscal year (the fiscal year of 2004 in this example). Thereby, it is possible to analyze whether the examinee who was not under treatment of diabetes in the first year (in the fiscal year of 2004 in this example) is now under treatment of diabetes (probability of diabetes treatment).

Then in a step 603 of calculating a difference between HbA1c and estimated HbA1c, the insulin secretory capacity evaluation equation creation unit 108 acquires a relational equation with the matched units of the fasting blood glucose level and the HbA1c value from the relational equation data of FIG. 5. The insulin secretory capacity evaluation equation creation unit 108 then substitutes the fasting blood glucose level included in the evaluation equation creation data extracted in the analysis data extraction step 602 into the relational equation thereby to calculate an estimated HbA1c value per examinee ID 201 for all the examinee IDs 201 included in the evaluation equation creation data. Further, the insulin secretory capacity evaluation equation creation unit 108 subtracts the calculated estimated HbA1c value from the HbA1c value included in the evaluation equation creation data thereby to calculate a difference value between HbA1c and estimated HbA1c per examinee ID 201.

Then in a corrected value determination step 604, the insulin secretory capacity evaluation equation creation unit 108 determines a corrected value by the ROC analysis (Receiver Operating Characteristic analysis) on the basis of a relationship between the difference value calculated in the step 603 of calculating a difference between an HbA1c value and an estimated HbA1c value, and the presence of diabetes treatment. Specifically, the insulin secretory capacity evaluation equation creation unit 108 generates a ROC curve on the basis of a relationship between the difference value between the HbA1c value and the estimated HbA1c value of the older fiscal year (the fiscal year of 2004 in this example) in the medical checkup data of two different fiscal years included in the evaluation equation creation data, and the probability of diabetes treatment in the medical checkup data of the later fiscal year (the fiscal year of 2009 in this example) thereby to determine a value with the maximum sensitivity+specificity as corrected value.

Then in an evaluation equation creation step 605, the insulin secretory capacity evaluation equation creation unit 108 creates an evaluation equation on the basis of the difference value between the HbA1c and the estimated HbA1c calculated in the step 603 of calculating a difference between an HbA1c value and an estimated HbA1c, and the corrected value determined in the corrected value determination step 604. Specifically, the evaluation equation is obtained by subtracting the corrected value from the difference between the HbA1c and the estimated HbA1c.

The insulin secretory capacity evaluation equation creation processing of FIG. 7 is now terminated. The created evaluation equations can be stored in the evaluation equation storage unit 123.

FIG. 8 illustrates exemplary evaluation equations created by the insulin secretory capacity evaluation equation creation unit 108. In the evaluation equation data illustrated in FIG. 8, an evaluation value can be calculated on the basis of the HbA1c value and the estimated HbA1c value of a subject to be analyzed. In evaluation equation data 700 illustrated in FIG. 8, evaluation equation 703 is stored per set of the units of HbA1c value 701 and fasting blood glucose level 702. Evaluation equation 703 indicates a relational equation created by the insulin secretory capacity evaluation equation creation unit 108, which is described in the form of [HbA1c value]−[estimated HbA1c value]−Th1 to Th3 by way of example. Th1 to Th3 in an evaluation equation is a corrected value calculated by the ROC analysis per set of the units of HbA1c value 701 and fasting blood glucose level 702.

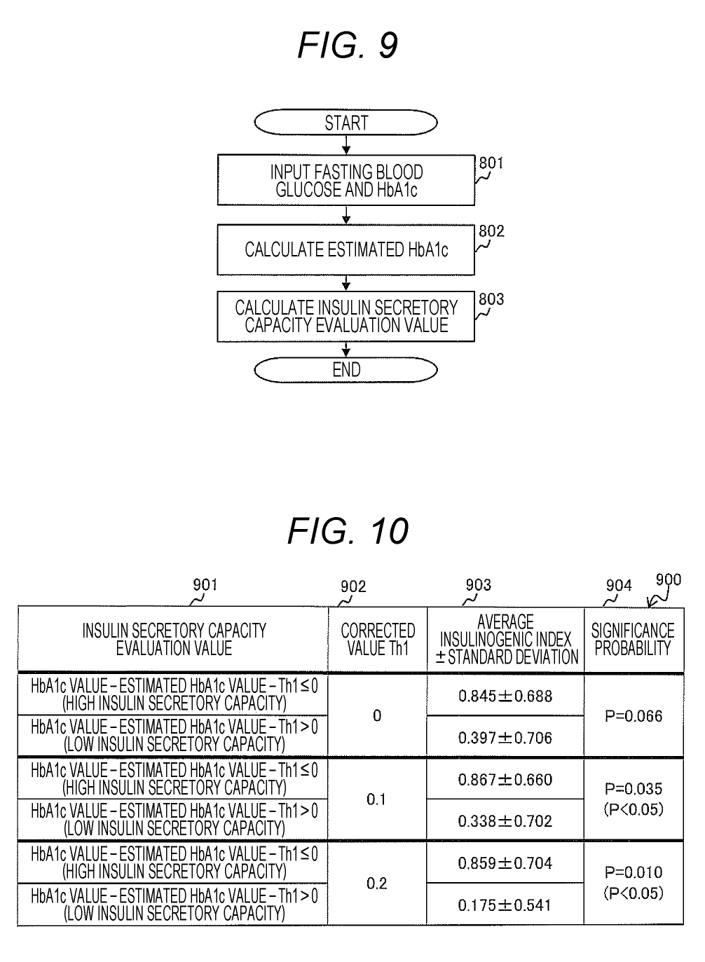

The insulin secretory capacity analysis device 101, which calculates the relational equations and the evaluation equations as described above, can calculate an evaluation value of the insulin secretory capacity of a subject to be analyzed according to the flowchart illustrated in FIG. 9, for example. When the processing in FIG. 9 is started, a fasting blood glucose/HbA1c input step 801 is first performed.

In the fasting blood glucose/HbA1c input step 801, the estimated HbA1c calculation unit 109 inputs at least the fasting blood glucose level and the HbA1c value of the subject to be analyzed in the input unit 102. At this time, information on weight change of the subject to be analyzed may be input.

Then in an estimated HbA1c calculation step 802, the estimated HbA1c calculation unit 109 first acquires the relational equation data stored in the relational equation storage unit 122. The estimated HbA1c calculation unit 109 then selects a relational equation with the matched units of the fasting blood glucose level and the HbA1c value of the subject to be analyzed input in the fasting blood glucose/HbA1c input step 801. The estimated HbA1c calculation unit 109 then substitutes the input fasting blood glucose level into the selected relational equation thereby to calculate an estimated HbA1c value for the subject to be analyzed.

Then in an insulin secretory capacity evaluation value calculation step 803, the insulin secretory capacity evaluation value calculation unit 110 first acquires the evaluation equation data stored in the evaluation equation storage unit 123. The insulin secretory capacity evaluation value calculation unit 110 then selects evaluation equation 703 with the matched units of the fasting blood glucose level and the HbA1c value of the subject to be analyzed from the evaluation equation data. The insulin secretory capacity evaluation value calculation unit 110 then substitutes the estimated HbA1c value calculated by the estimated HbA1c calculation unit 109 and the HbA1c value input in the fasting blood glucose/HbA1c input step 801 into the selected evaluation equation thereby to calculate an evaluation value for the insulin secretory capacity. According to the thus-calculated evaluation equation, it is possible to determine that the insulin secretory capacity is low when the calculated evaluation value is positive and that the insulin secretory capacity is high when the calculated evaluation value is negative. It is possible to acquire the information on the insulin secretory capacity (information that the insulin secretory capacity is high or low) by comparing the evaluation value for the insulin secretory capacity calculated by the insulin secretory capacity evaluation value calculation unit 110 with the preset reference value depending on the definition of the evaluation equation. Further, the information on the insulin secretory capacity of the subject to be analyzed can be output to the output unit 103.

The insulin secretory capacity calculation processing is now terminated. The insulin secretory capacity can be simply evaluated on the basis of the fasting blood glucose level and the HbA1c value checked in general medical checkup or complete medical checkup through the processing. That is, with the insulin secretory capacity analysis device 101 according to the present invention, the insulin concentration, which is not checked in general medical checkup or complete medical checkup, does not need to be measured and a blood specimen does not need to be taken and analyzed twice while an examinee is hungry and after the glucose tolerance test. In this way, the insulin secretory capacity analysis device 101 according to the present invention can determine the insulin secretory capacity very simply. Further, the insulin secretory capacity analysis device 101 according to the present invention preferably includes, though not illustrated, a management necessity determination unit for determining the necessity of management of diabetes of a subject to be analyzed on the basis of weight information and an insulin secretory capacity evaluation value when the information on weight change (weight information) of the subject to be analyzed is input by the input unit 102. When the insulin secretory capacity of the subject to be analyzed decreases and the weight information indicates an increase in weight, the management necessity determination unit determines that management for prevention of diabetes is required for the subject to be analyzed.

FIG. 10 illustrates result 900 in which the insulin secretory capacity evaluation value calculated by the insulin secretory capacity analysis device 101 according to the present invention is evaluated by a conventional insulinogenic index. That is, FIG. 10 illustrates the results in which average insulinogenic index±standard deviation 903 is calculated per corrected value Th1 902 by dividing insulin secretory capacity evaluation value 901 into two groups of positive and negative. Further, T-test is performed on a difference between the average values of the two groups thereby to indicate calculated significance probability 904. FIG. 10 illustrates the evaluation results using the data of 24 examinees, which indicate that the average value of the insulinogenic index is lower in the group of positive of the insulin secretory capacity evaluation value. The significance probability is less than 0.05 at the corrected value Th1 of 0.1 or 0.2, which is a statistically significant difference. As described above, it is demonstrated that the insulin secretory capacity evaluation value calculated by the insulin secretory capacity analysis device 101 according to the present invention can evaluate the insulin secretory capacity of a subject to be analyzed with similar accuracy to the system for evaluating the insulin secretory capacity by use of a conventional insulinogenic index.

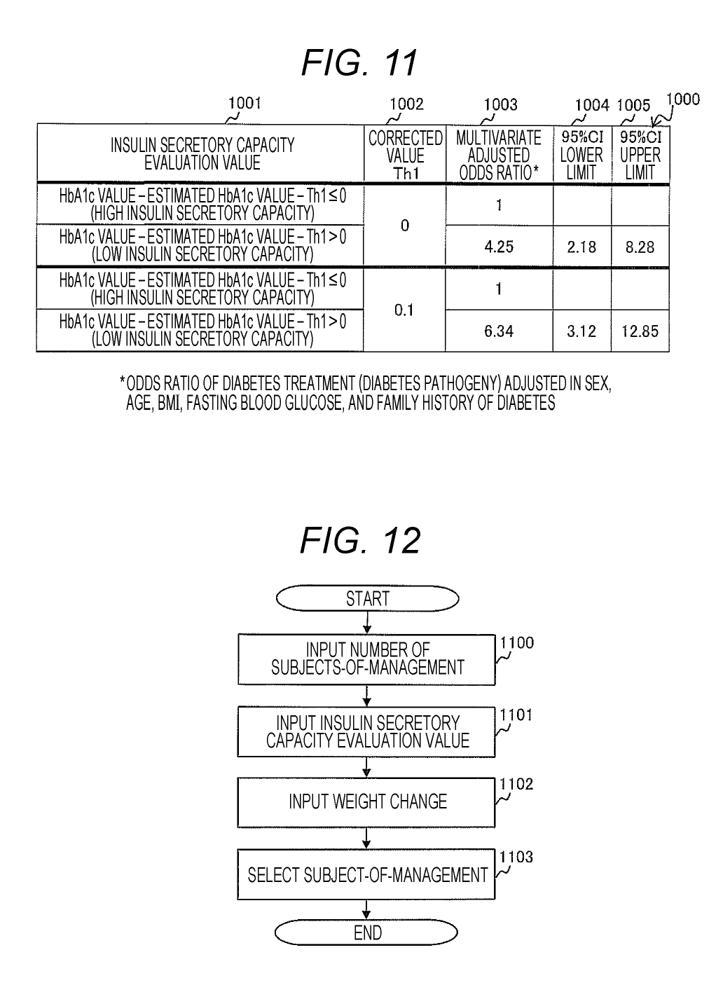

FIG. 11 illustrates result 1000 in which the insulin secretory capacity evaluation value calculated by the insulin secretory capacity analysis device 101 according to the present invention is evaluated by the presence of diabetes treatment five years later. FIG. 11 illustrates the results in which multivariate adjusted odds ratio 1003 of diabetes treatment (pathogeny of diabetes), and lower limit 1004 and upper limit 1005 of 95% confidence interval (95% CI) are calculated per corrected value Th1 1002 by dividing insulin secretory capacity evaluation value 1001 into two groups of positive and negative. The multivariate adjusted odds ratio indicates an odds ratio of the group of positive assuming the group of negative at 1 in insulin secretory capacity evaluation value 1001, and is a value adjusted by sex, age, BMI, fasting blood glucose, and family history of diabetes, which is other covariate related to pathogeny of diabetes. The group of positive in insulin secretory capacity evaluation value 1001 indicates that the probability of diabetes treatment five years later is 4.25 times higher than the group of negative and the 95% CI lower limit exceeds 1 on the basis of multivariate adjusted odds ratio 1003 and 95% CI lower limit 1004 of FIG. 11, which means a significant result. It is clear from the results illustrated in FIG. 11 that the insulin secretory capacity can be simply evaluated and a future risk of diabetes can be evaluated by use of the insulin secretory capacity evaluation value calculated by the insulin secretory capacity analysis device 101 according to the present invention.

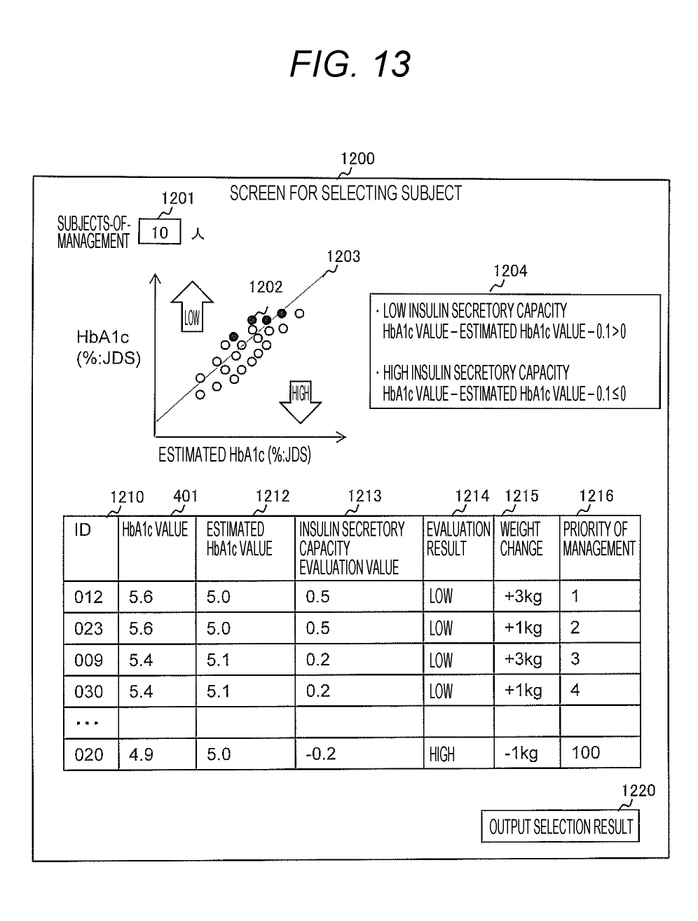

The insulin secretory capacity analysis device 101 according to the present invention can further perform a subject-of-management selection processing by the subject-of-management selection unit 111. The insulin secretory capacity analysis device 101 can perform the subject-of-management selection processing in the flowchart illustrated in FIG. 12, for example. FIG. 13 illustrates an exemplary screen for selecting a subject used for the subject-of-management selection processing.

In the subject-of-management selection processing, as illustrated in FIG. 12, a number-of-subjects input step 1100 is first performed.

In the number-of-subjects input step 1100, the number of subjects of management is input by the input unit 102 into number-of-subjects input column 1201 in the subject selection screen of FIG. 13 in consideration of budget for management or the like.

The subject selection screen 1200 illustrated in FIG. 13 displays therein number-of-subjects input column 1201, distribution diagram 1202 of HbA1c value and estimated HbA1c value of candidate subject, graph 1203 indicating an insulin secretory capacity evaluation equation, and insulin secretory capacity high/low determination reference 1204. The subject selection screen 1200 illustrated in FIG. 13 displays therein subject candidate ID 1210, HbA1c 401, estimated HbA1c 1212, insulin secretory capacity evaluation value 1213, insulin secretory capacity high/low evaluation result 1214, weight change 1215, and management priority 1216 in the table form. The subject selection screen 1200 illustrated in FIG. 13 displays therein a selection result output button 1220 for outputting a subject-of-management selection result.

Then in an insulin secretory capacity evaluation value input step 1201, the subject-of-management selection unit 111 inputs as many insulin secretory capacity evaluation values calculated by the insulin secretory capacity evaluation value calculation unit 110 as the subjects in the column of insulin secretory capacity evaluation value 1213. The input insulin secretory capacity evaluation values evaluate the insulin secretory capacity, which is displayed in the table form together with HbA1c 401 and estimated HbA1c 1212 per ID 1210 as illustrated in FIG. 13.

Then in a weight change input step 1102, the subject-of-management selection unit 111 inputs as many weight changes as the subjects by the input unit 102. The input weight changes are displayed in the table form per ID 1210 as illustrated in FIG. 13.

Then in a subject-of-management selection step 1103, the subject-of-management selection unit 111 selects as many subjects-of-management as the number of subjects of management input in the number-of-subjects input step 1100 on the basis of the insulin secretory capacity evaluation values input in the insulin secretory capacity evaluation value input step 1101 and the weight changes input in the weight change input step 1102. Specifically, the management priority of a subject for which the insulin secretory capacity evaluation value is high and the weight change is large is increased, and as many subjects as the number of subjects of management are selected. FIG. 13 indicates that a subject with low management priority 1216 has a high insulin secretory capacity evaluation value and a large weight change. When the subjects of management are determined, the selection result output button 1220 in FIG. 13 is pressed to output a list of subjects.

As described above, the insulin secretory capacity analysis device 101 according to the present invention can complete the subject-of-management selection processing by the subject-of-management selection unit 111.

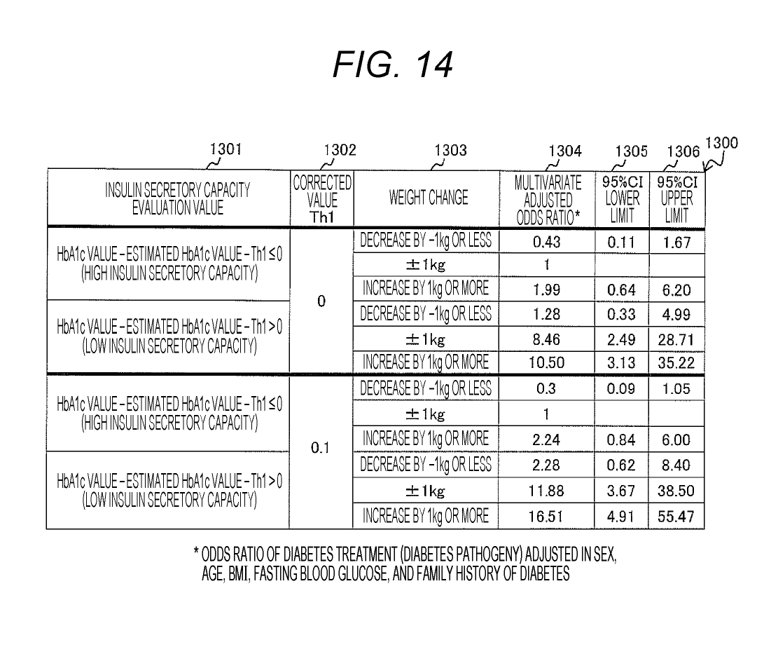

FIG. 14 illustrates the results of the evaluated presence of diabetes treatment five years later in combination of the insulin secretory capacity evaluation value and the weight change calculated by the insulin secretory capacity analysis device 101 according to the present invention. FIG. 14 illustrates the results in which multivariate adjusted odds ratio 1304 of diabetes treatment (pathogeny of diabetes), and lower limit 1305 and upper limit 1306 of 95% confidence interval (95% CI) are calculated per corrected value Th1 1302 and weight change 1303 with the two divided groups of positive and negative of insulin secretory capacity evaluation value 1301. The multivariate adjusted odds ratio indicates an odds ratio in each group when the group with insulin secretory capacity evaluation value 1301 of negative and weight change 1303 of±1 kg is assumed at 1, and indicates a value adjusted by sex, age, BMI, fasting blood glucose level, and family history of diabetes, which is other covariate related to pathogeny of diabetes. The groups for which insulin secretory capacity evaluation value 1301 is positive and weight change is an increase by 1 kg or more indicate that the probability of diabetes treatment five years later is 10.5 times higher and the 95% CI lower limit exceeds 1 on the basis of multivariate adjusted odds ratio 1304 and 95% CI lower limit 1305 of FIG. 14, which is a significant result.

In this way, the insulin secretory capacity analysis device 101 according to the present invention can derive a result capable of evaluating a future risk of diabetes in combination of the insulin secretory capacity evaluation value calculated by the insulin secretory capacity evaluation value calculation unit 110 and the information on weight change. The insulin secretory capacity analysis device 101 according to the present invention can appropriately select a subject of management of diabetes in combination of the insulin secretory capacity evaluation value calculated by the insulin secretory capacity evaluation value calculation unit 110 and the information on weight change.

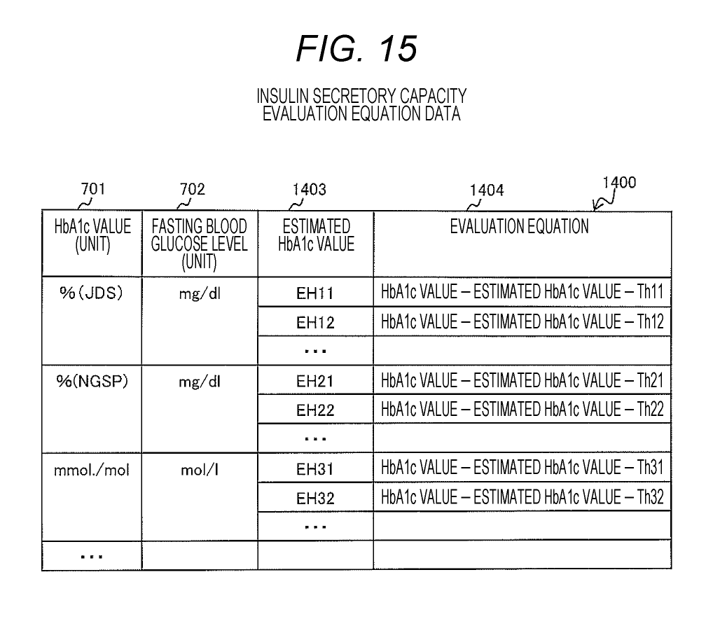

As described above, the insulin secretory capacity evaluation equation creation unit 108 calculates a corrected value on the basis of a relationship between the difference value between an HbA1c value and an estimated HbA1c value and the presence of diabetes treatment thereby to create an evaluation equation of FIG. 8 in the insulin secretory capacity analysis device 101, but the evaluation equation may be created in other method, not limited to the method. For example, a relationship between a difference value between an HbA1c value and an estimated HbA1c value and the presence of diabetes treatment is subjected to ROC analysis and a corrected value per estimated HbA1c value is calculated thereby to create an evaluation equation per estimated HbA1c value in the insulin secretory capacity analysis device 101. That is, in this example, for the created evaluation equations, evaluation equation 1404 per estimated HbA1c value 1403 is stored per set of the units of HbA1c value 701 and fasting blood glucose level 702 as illustrated in FIG. 15. In FIG. 15, EH11 and EH12 indicate a corrected value calculated for a set of the unit of HbA1c value 701 of “% (JDS)” and the unit of fasting blood glucose level 702 of “mg/dl.” In this way, an evaluation equation is created by use of a different corrected value depending on an estimated HbA1c value thereby to evaluate the insulin secretory capacity with higher accuracy. Consequently, an evaluation equation is used by use of a different corrected value depending on an estimated HbA1c value thereby to evaluate a future risk of diabetes with higher accuracy.

As described above, the insulin secretory capacity analysis device 101 selects a subject of management of diabetes on the basis of the insulin secretory capacity evaluation value and the weight change, but a subject of management may be selected not in consideration of weight change, or a subject of management may be selected by use of other information and the insulin secretory capacity evaluation value instead of weight change.

With the insulin secretory capacity analysis system in which the insulin secretory capacity analysis device 101 is connected to a terminal having a dataset including at least a fasting blood glucose level and an HbA1c value of a subject to be analyzed, the fasting blood glucose level and the HbA1c value of the subject to be analyzed are input from the terminal into the insulin secretory capacity analysis device 101 thereby to analyze an insulin secretory capacity of the subject to be analyzed. Herein, the terminal may be a server computer storing the medical checkup results therein or may be a household blood glucose meter, for example. For example, with the insulin secretory capacity analysis system using a household blood glucose meter, the HbA1c value and the fasting blood glucose level are simply measured by the meter thereby to grasp the insulin secretory capacity of a subject. The insulin secretory capacity analysis system is used for daily insulin treatment on the basis of an insulin secretory capacity evaluation value.

### REFERENCE SIGNS LIST

101 insulin secretory capacity analysis terminal
102 input unit
103 output unit
104 CPU
105 memory
106 storage medium
107 relational equation creation unit
108 insulin secretory capacity evaluation equation creation unit
109 estimated HbA1c calculation unit
110 insulin secretory capacity evaluation value calculation unit
111 subject-of-management selection unit
120 database
121 medical checkup information recording unit
122 relational equation storage unit
123 evaluation equation storage unit
124 subject-of-management storage unit

1. An insulin secretory capacity analysis device comprising:
an input unit that inputs at least a fasting blood glucose level and an HbA1c value;
an estimated HbA1c calculation unit that calculates an estimated HbA1c value on the basis of the input fasting blood glucose level and HbA1c value; and
an insulin secretory capacity evaluation value calculation unit that calculates an insulin secretory capacity evaluation value on the basis of the HbA1c value input by the input unit and the estimated HbA1c value calculated by the estimated HbA1c calculation unit.

2. The insulin secretory capacity analysis device according to claim 1,
wherein the estimated HbA1c calculation unit calculates an estimated HbA1c value on the basis of the input fasting blood glucose level and HbA1c value by use of a relational equation of the fasting blood glucose level and the HbA1c value created on the basis of a dataset including the fasting blood glucose level and the HbA1c value of a plurality of examinees.

3. The insulin secretory capacity analysis device according to claim 2,
wherein the regression analysis is made assuming the HbA1c value as objective variable and the fasting blood glucose level as explanatory variable thereby to create the relational equation.

4. The insulin secretory capacity analysis device according to claim 1,
wherein the insulin secretory capacity evaluation value calculation unit calculates the insulin secretory capacity evaluation value on the basis of a difference between the HbA1c value input by the input unit and the estimated HbA1c value calculated by the estimated HbA1c calculation unit.

5. The insulin secretory capacity analysis device according to claim 1, further comprising:
an output unit that outputs information on an insulin secretory capacity by comparing the insulin secretory capacity evaluation value calculated by the insulin secretory capacity evaluation value calculation unit with a reference value.

6. The insulin secretory capacity analysis device according to claim 1, further comprising:
a management necessity determination unit that determines the necessity of management of diabetes on the basis of weight information input by the input unit and the insulin secretory capacity evaluation value calculated by the insulin secretory capacity evaluation value calculation unit.

7. The insulin secretory capacity analysis device according to claim 1, further comprising:
a medical checkup data storage unit that stores therein a dataset including a fasting blood glucose level and an HbA1c value of a plurality of examinees; and
a subject-of-management selection unit that selects a subject of management of diabetes from among the dataset stored in the medical checkup data storage unit on the basis of the weight information input by the input unit and the insulin secretory capacity evaluation value calculated by the insulin secretory capacity evaluation value calculation unit.

8. An insulin secretory capacity analysis system comprising:
the insulin secretory capacity analysis device according to claim 1; and
a terminal having a dataset including at least a fasting blood glucose level and an HbA1c value of a subject to be analyzed,
wherein the dataset of a subject to be analyzed is input from the terminal into the insulin secretory capacity analysis device, and an insulin secretory capacity of the subject to be analyzed is analyzed by the insulin secretory capacity analysis device.

9. The insulin secretory capacity analysis system according to claim 8,
wherein the terminal is a measurement instrument for measuring a fasting blood glucose level of a subject to be analyzed and/or for measuring an HbA1c value.

10. An insulin secretory capacity analysis method comprising the steps of:
inputting a fasting blood glucose level and an HbA1c value;
calculating an estimated HbA1c value on the basis of the input fasting blood glucose level and HbA1c value; and
calculating an insulin secretory capacity evaluation value on the basis of the input HbA1c value and the calculated estimated HbA1c value.
